(19) United States
(12) Patent Application Publication
(10) Pub. No.: US 2022/0280494 A1
(43) Pub. Date: Sep. 8, 2022

(54) COMPOSITIONS FOR SMALL MOLECULE THERAPEUTIC AGENT COMPOUNDS

(71) Applicant: Delpor, Inc., Brisbane, CA (US)

(72) Inventor: Gregory A. Watkins, Oakland, CA (US)

(21) Appl. No.: 17/637,755

(22) PCT Filed: Aug. 27, 2020
(86) PCT No.: PCT/US2020/048277

§ 371 Date: Feb. 23, 2022

Related U.S. Application Data
(60) Provisional application No. 62/893,124, filed on Aug. 28, 2019.

(51) Int. Cl. A61K 31/4468; A61K 31/451; A61K 31/485; A61K 45/06; A61K 47/12

(52) U.S. Cl. CPC A61K 31/4468; A61K 31/451; A61K 31/485; A61K 45/06; A61K 47/12

(57) ABSTRACT

A composition comprising a small molecule therapeutic agent and a polyprotic acid compound or a mixture of two monoprotic acid compounds is described. The polyprotic acid is present in an amount that (i) is equal to or less than the molar amount of the therapeutic agent and (ii) provides a total number of acid hydrogens that exceeds the stoichiometric equivalent for the molar amount of therapeutic agent. The two or more monoprotic acids are present such that each monoprotic acid is in an amount that is equal to or less than the molar amount of the therapeutic agent and together the two or more monoprotic acids provide a total number of acid hydrogens that exceeds the stoichiometric equivalent for the molar amount of therapeutic agent. The polyprotic acid or mixture of monoprotic acids provide a composition that delivers the therapeutic agent for a sustained period of time.

### TECHNICAL FIELD

The subject matter described herein relates to compositions and formulations for a small molecule therapeutic agent, and to drug delivery devices comprising the compositions and formulations for controlled, sustained delivery of the small molecule therapeutic agent.

### BACKGROUND

Important classes of small molecule drugs exhibit poor water solubility at neutral pH. Although this property may favor tissue penetration by transmembrane diffusion, particularly for drugs that target the central nervous system, it complicates the development of injectable or implantable sustained delivery systems which rely on passive diffusion as the primary drug release mechanism. For example, a hydrophobic drug with greatly reduced water solubility may not be able to create a concentration gradient across a membrane, porous partition, or other phase boundary sufficient to drive adequate efflux from a reservoir containing an aqueous suspension of the drug. Many insoluble drugs are weak organic bases (i.e., molecules that include at least one functional group such as a primary, secondary, or tertiary amine; aniline, amidine, or guanidine; or a nitrogen-bearing heterocyclic ring such as pyridine, quinoline, imidazole, thiazole, triazole, or tetrazole), and their water solubility improves upon protonation; i.e., when they are converted into a salt. Many drugs that target the central nervous system fall into this category, including antipsychotics (e.g., risperidone, paliperidone, olanzapine, and haloperidol), antidepressants (e.g., citalopram, escitalopram, and buspirone), opioid agonists and antagonists (e.g., buprenorphine, naloxone, naltrexone, and 4-phenylpiperidines such as fentanyl and meperidine); antimigraine agents (e.g., rizatriptan, naratriptan, sumatriptan, and zolmitriptan); antiemetics (e.g., granisetron, ondansetron, and other serotonin receptor antagonists); anticonvulsants (e.g., perampanel); dopaminergic antiparkinsonian agents (e.g., pramipexole, ropinirole, rotigotine, cabergoline, and bromocriptine); acetylcholinesterase inhibitors (e.g., rivastigmine and donepezil); skeletal muscle relaxants (e.g., tizanidine and cyclobenzaprine); nicotine agonists or partial agonists (e.g., varenicline) and VMAT2 inhibitors (e.g., tetrabenazine and deutetrabenazine). Examples of hydrophobic base drugs that target receptors, cells, or tissues outside of the central nervous system include alpha blockers (e.g., prazosin), cardiac inotropic agents (e.g., dobutamine), antimalarials (e.g., primaquine and mefloquine), aromatase inhibitors (e.g., anastrazole and letrozole), antiestrogens (e.g., tamoxifen and raloxifene), phosphodiesterase inhibitors (e.g., vardenafil), and immunomodulators (e.g., fingolimod).

Although salts formed between such drugs and a canonical acid may have improved solubility in water, they are unstable and susceptible to hydrolysis at pH values approaching or exceeding the pKa of the protonated drug, which is typically greater than 7. This process complicates diffusion-mediated drug delivery through an implant or depot (i.e., a delivery mechanism that lacks an active pumping mechanism or a complicated semi-permeable membrane architecture to regulate release), since efflux of drug from the formulation must be coupled to a constant influx of buffering species from physiological fluids. Additionally, if the salt form of a drug is to be delivered by way of an implantable device, the composition must contain a volume of active drug sufficient to provide a therapeutic benefit for a given dosing period; yet, the total formulation volume should be minimized in order to limit the size (and therefore, the degree of invasiveness) of the final implant design. Compositions and devices that address these, and other, complications related to sustained and controlled delivery of small molecule therapeutic agents that are weak organic bases, are needed.

### BRIEF SUMMARY

The following aspects and embodiments thereof described and illustrated below are meant to be exemplary and illustrative, not limiting in scope.

In one aspect, compositions comprising a molar amount of a small molecule therapeutic agent that (i) has a water solubility at 25° C. of less than about 1 g/L and (ii) is a weak base (i.e., possessing a conjugate acid with a pKa between 6 and 9) and a polyprotic acid or a combination of two or more monoprotic acids is provided.

In another aspect, the composition comprises a molar amount of the therapeutic agent and an organic polyprotic acid in an amount that is (i) equal to or less than the molar amount of the therapeutic agent and (ii) provides a total number of acid hydrogens that exceeds the stoichiometric equivalent for the molar amount of therapeutic agent.

In another aspect, the composition comprises a molar amount of the therapeutic agent and an inorganic polyprotic acid in an amount that is (i) equal to or less than the molar amount of the therapeutic agent and (ii) provides a total number of acid hydrogens that exceeds the stoichiometric equivalent for the molar amount of therapeutic agent.

In another aspect, the composition comprises a molar amount of the therapeutic agent and a mixture of two or more acids, wherein the mixture comprises at least one organic polyprotic acid in an amount that is (i) equal to or less than the molar amount of the therapeutic agent and (ii) provides a total number of acid hydrogens that exceeds the stoichiometric equivalent for the molar amount of therapeutic agent.

In another aspect, the composition comprises a molar amount of the therapeutic agent and a mixture of two or more acids, wherein the mixture comprises at least one inorganic polyprotic acid in an amount that is (i) equal to or less than the molar amount of the therapeutic agent and (ii) provides a total number of acid hydrogens that exceeds the stoichiometric equivalent for the molar amount of therapeutic agent.

In still another aspect, the composition comprises a molar amount of the therapeutic agent and a dendrimer capped with an acidic functional group, the dendrimer in an amount that is (i) between about 0.25-1. of the molar amount of therapeutic agent and (ii) provides a total number of acid hydrogens that exceeds the stoichiometric equivalent for the molar amount of therapeutic agent.

In yet another aspect, the composition comprises a molar amount of the therapeutic agent and a mixture of acidic excipients including at least one dendrimer capped with an acidic functional group, the dendrimer in an amount that is (i) between about 0.25-1. of the molar amount of therapeutic agent and (ii) provides a total number of acid hydrogens that exceeds the stoichiometric equivalent for the molar amount of therapeutic agent.

In yet another aspect, the composition comprises a molar amount of the therapeutic agent and a mixture of two or more monoprotic acids, wherein each monoprotic acid in the mixture is in an amount that is equal to or less than the molar amount of the therapeutic agent and wherein the two or more monoprotic acids provide a total number of acid hydrogens that exceeds the stoichiometric equivalent for the molar amount of therapeutic agent.

In some embodiments, the organic acid compound (i) has a water solubility at room temperature of less than about 20 g/L (ii) maintains a pH of the suspension in its environment of use of between 3-6.5 for a period of at least about 30 days, and/or (iii) has a molecular weight less than or equal to 500 grams per mole.

In some embodiments, the composition is an aqueous suspension. The aqueous suspension comprises a molar amount of a small molecule therapeutic agent that (i) has a water solubility at 25° C. of less than about 1 g/L and (ii) is a weak base (i.e., possessing a conjugate acid with a pKa between 5 and 9) and a polyprotic acid compound in an amount that is (i) equal to or less than the molar amount of the therapeutic agent and (ii) provides a total number of acid hydrogens that exceeds the stoichiometric equivalent for the molar amount of therapeutic agent.

In one aspect, the therapeutic agent becomes more soluble upon protonation.

In another aspect, a composition comprising an aqueous suspension is provided. The aqueous suspension comprises a small molecule therapeutic agent that (i) has a water solubility at 25° C. of less than about 1 g/L and (ii) becomes more soluble upon protonation, and a polyprotic acid compound in an amount that is (i) equal to or less than the molar amount of the therapeutic agent and (ii) provides a total number of acid hydrogens that exceeds the stoichiometric equivalent for the molar amount of therapeutic agent. The polyprotic acid compound, in one embodiment, is a mixture of organic acid compounds that comprises at least one polyprotic acid or at least one dendrimer.

In one embodiment, the aqueous suspension is a heterogeneous mixture comprising the small molecule therapeutic agent and a polyprotic organic acid compound, where the organic acid compound sufficiently dissolves to maintain the pH of the heterogeneous solution in its environment of use at a value equal to or less than physiological pH (˜7.4) for the stated period. In one embodiment, the environment of use is in vivo. In another embodiment, the environment of use is in vitro in a release medium maintained at a controlled temperature, e.g., 37° C.

In one embodiment, the aqueous suspension is a heterogeneous mixture comprising the small molecule therapeutic agent and a mixture of organic acids containing at least one polyprotic acid, where the organic acids are selected to dissolve at a rate that maintains the pH of the aqueous suspension (or of the heterogeneous mixture) in its environment of use at a value equal to or less than physiological pH (˜7.4) for the stated period. In one embodiment, the environment of use is in vivo. In another embodiment, the environment of use is in vitro in a release medium maintained at a controlled temperature, e.g., 37° C.

In another embodiment, the organic acid compound is crystalline and has a melting temperature of more than about 37° C.

In one embodiment, the small molecule therapeutic agent is selected to be a typical or atypical antipsychotic, such as risperidone, olanzapine, paliperidone, aripiprazole, brexpiprazole, asenapine, lurasidone, cariprazine, or haloperidol.

In one embodiment, the small molecule therapeutic agent is selected from opioid agonists and antagonists (e.g., buprenorphine, naloxone, naltrexone, and 4-phenylpiperidines such as fentanyl and meperidine); antimigraine agents (e.g., rizatriptan, naratriptan, sumatriptan, and zolmitriptan); antiemetics (e.g., granisetron, ondansetron, and other serotonin receptor antagonists); anticonvulsants (e.g., perampanel); dopaminergic antiparkinsonian agents (e.g., pramipexole, ropinirole, cabergoline, and bromocriptine); acetylcholinesterase inhibitors (e.g., rivastigmine and donepezil); skeletal muscle relaxants (e.g., tizanidine and cyclobenzaprine); nicotine agonists or partial agonists (e.g., varenicline); immunomodulating agents (e.g., fingolimod), and/or VMAT2 inhibitors (e.g., tetrabenazine and deutetrabenazine).

In another embodiment, the small molecule therapeutic agent is selected from opioid agonists and antagonists, anti-Parkinsonian agents, anti-migraine agents, agents that act as skeletal muscle relaxants, anti-emetics, and/or immunomodulators for treating Multiple sclerosis. Other embodiments include any one or any combination of classes of therapeutic agents and/or the therapeutic agents discloses herein.

In another embodiment, the therapeutic agent is an organic base structurally derived from a fatty acid, such as fingolimod.

In another embodiment, the therapeutic agent is a cardiac inotropic agent such as dobutamine.

In yet another embodiment, the therapeutic agent is an anti-hypertensive drug such as prazosin.

In one embodiment, the therapeutic agent is an anti-malarial drug such as primaquine or mefloquine.

In yet another embodiment, the therapeutic agent is an aromatase inhibitor such as anastrazole or letrozole.

In one embodiment, the therapeutic agent has antiestrogen activity, such as tamoxifen or raloxifene.

In one embodiment, the therapeutic agent is a benzothiazole, such as riluzole.

In one embodiment, the therapeutic agent is an alpha-blocker, such as prazosin or terazosin, or a beta blocker, such as metoprolol or propranolol.

In one embodiment, the aqueous suspension comprises, or is manufactured with, an organic acid suspended into a water-based solution, such as an aqueous buffered solution.

In another embodiment, the aqueous suspension comprises, or is manufactured with, a pre-made salt formed between the molar amount of the therapeutic agent and at least one polyprotic acid, such that the polyprotic acid is in an amount that is (i) equal to or less than the molar amount of the therapeutic agent and (ii) provides a total number of acid hydrogens that exceeds the stoichiometric equivalent for the molar amount of therapeutic agent.

In another embodiment, the therapeutic agent and the at least one polyprotic acid, or more than one monoprotic acid, or mixture with at least one dendrimer are mixed by dissolution into an organic solvent such as methanol, ethanol, 1-propanol, 2-propanol, tert-butanol, acetone, 2-butanone, or ethyl acetate, followed by concentration of the intermediate solution to dryness to yield a salt form of the therapeutic agent.

In one embodiment, the therapeutic agent and less than molar amount of a polyprotic acid are mixed by dissolution into an organic solvent such as methanol, ethanol, 1-propanol, 2-propanol, tert-butanol, acetone, 2-butanone, or ethyl acetate, followed by concentration of the intermediate solution to dryness to yield a mixture of salt forms of the therapeutic agent.

In another embodiment, the therapeutic agent is mixed with (i) a mixture of organic acids containing at least one polyprotic acid in an amount that is less than the molar amount of the therapeutic agent and (ii) an organic solvent such as methanol, ethanol, 1-propanol, 2-propanol, tert-butanol, acetone, 2-butanone, or ethyl acetate, followed by concentration of the intermediate solution to dryness to yield a mixture of salt forms of the therapeutic agent.

In another embodiment, the therapeutic agent is mixed with (i) a mixture of organic acids containing more than one monoprotic acid, wherein each monoprotic acid in the mixture is in an amount that is equal to or less than the molar amount of the therapeutic agent and wherein the two or more monoprotic acids provide a total number of acid hydrogens that exceeds the stoichiometric equivalent for the molar amount of therapeutic agent, and (ii) an organic solvent such as methanol, ethanol, 1-propanol, 2-propanol, tert-butanol, acetone, 2-butanone, or ethyl acetate, followed by concentration of the intermediate solution to dryness to yield a mixture of salt forms of the therapeutic agent.

In one embodiment, the formulation comprises an organic acid that is an aromatic carboxylic acid. Exemplary acids, in one embodiment, are those having a carboxylic acid group bound to an unsubstituted benzene or pyridine ring. In one embodiment, the carboxylic acid is selected from the group consisting of benzoic acid, picolinic acid, nicotinic acid, and isonicotinic acid.

In another embodiment, the formulation comprises a carboxylic acid having a benzene ring and one electron-donating group. In another embodiment, the carboxylic acid has antioxidant properties.

In still another embodiment, the formulation comprises a carboxylic acid selected from the group consisting of o-anisic acid, m-anisic acid, p-anisic acid, p-aminobenzoic acid (PABA), o-aminobenzoic acid (anthranilic acid), o-toluic acid, m-toluic acid, p-toluic acid and salicylic acid.

In another embodiment, the formulation comprises a carboxylic acid having one benzene ring and two electron donating groups. In another embodiment, the carboxylic acid has antioxidant properties. In one embodiment, and by way of example, the carboxylic acid is vanillic acid.

In yet another embodiment, the formulation comprises a carboxylic acid having at least two carboxylic acid groups bonded to a benzene ring. In one embodiment, and by way of example, the carboxylic acid is phthalic acid.

In yet another embodiment, the formulation comprises a carboxylic acid having a carboxylic acid group bonded to a naphthalene or quinoline ring. In one embodiment, and by way of example, the carboxylic acid is selected from the group consisting of 1-naphthoic acid, 2-naphthoic acid, quinaldic acid, 3-quinolinecarboxylic acid, 4-quinolinecarboxylic acid, 5-quinolinecarboxylic acid, 6-quinolinecarboxylic acid, 7-quinolinecarboxylic acid, and 8-quinolinecarboxylic acid.

In another embodiment, the formulation comprises a carboxylic acid having an aromatic ring bearing an electron-donating group selected from the group consisting of hydroxy, methoxy, amino, alkylamino, dialkylamino, and alkyl. In one embodiment, and by way of example, the carboxylic acid is selected from the group consisting of 6-hydroxy-2-naphthoic acid, 6-hydroxy-3-naphthoic acid, 8-hydroxy-2-quinolinecarboxylic acid and 8-hydroxy-7-quinolinecarboxylic acid.

In yet another embodiment, the formulation comprises a carboxylic acid having one or two carboxylic acid groups directly bonded to a biphenyl ring system. In one embodiment, and by way of example, the carboxylic acid is selected from the group consisting of 2-phenylbenzoic acid, 3-phenylbenzoic acid, 4-phenylbenzoic acid and diphenic acid.

In yet another embodiment, the formulation comprises a carboxylic acid having one additional electron donating substituent on the biphenyl carboxylic acid moiety. In one embodiment, and by way of example, the carboxylic acid is selected from the group consisting of 4′-hydroxy-4-biphenylcarboxylic acid, 4′-hydroxy-2-biphenylcarboxylic acid, 4′-methyl-4-biphenylcarboxylic acid, 4′-methyl-2-biphenylcarboxylic acid, 4′-methoxy-4-biphenylcarboxylic acid, and 4′-methoxy-2-biphenylcarboxylic acid.

In still another embodiment, the formulation comprises a carboxylic acid having a carboxylic acid functional group separated from a benzene, pyridine, naphthalene, quinoline, or coumarin ring by a chain of 1-4 saturated carbon atoms. In one embodiment, and by way of example, the carboxylic acid is phenylacetic acid, 3-phenylpropionic acid, or 7-hydroxycoumarin-4-acetic acid.

In another embodiment, the formulation comprises a carboxylic acid that is an aliphatic dicarboxylic acid with a 4-8 carbon chain separating the carboxylic acid groups. In one embodiment, and by way of example, the carboxylic acid is selected from the group consisting of adipic acid ((CH₂)₄(COOH)₂), pimelic acid (HO₂C(CH₂)₅CO₂H), suberic acid (HO₂C(CH₂)₆CO₂H), azelaic acid (HO₂C(CH₂)₇CO₂H), and sebacic acid (HO₂C(CH₂)₈CO₂H).

In another embodiment, the formulation comprises a polymer, dendrimer, peptide, or protein/In one embodiment, the formulation excludes polylactic acid-glycolic acid polymer and copolymers, and in another embodiment excludes polylactic acid-glycolic acid polymer and copolymers with a solubility less than or equal to about 20 g/L and an isoelectric point less than 7.

In another embodiment, the formulation comprises a carboxylic acid that is an unsaturated or polyunsaturated dicarboxylic acid containing 4-10 carbons. In one embodiment, and by way of example, the carboxylic acid is selected from the group consisting of fumaric acid, trans, trans-muconic acid, cis, trans-muconic acid, and cis, cis-muconic acid.

In other embodiments, the formulation comprises a carboxylic acid that is a cis-cinnamic acid or a trans-cinnamic acid. In still other embodiments, the carboxylic acid is a trans-cinnamic acid with one or two electron-donating groups selected from hydroxy, methoxy, amino, alkylamino, dialkylamino, or alkyl groups. In yet other embodiments, the trans-cinnamic acid is selected from the group consisting of o-coumaric acid, m-coumaric acid, p-coumaricacid, o-methylcinnamic acid, m-methylcinnamic acid, p-methylcinnamic acid, o-methoxycinnamic acid, m-methoxycinnamic acid, p-methoxycinnamic acid, and ferulic acid.

In one embodiment, the formulation comprises an organic acid that is a phenol or a naphthol substituted with between about 2-5 electron-withdrawing groups selected from F, Cl, Br, I, CN, and NO₂. In one embodiment, and by way of example, the organic acid is pentafluorophenol or 2,4-dinitrophenol.

In another embodiment, the formulation comprises an organic acid that is a 1,3-dicarbonyl compound containing an acidic CH or NH bond (pKa<8). In one embodiment, and by way of example, the organic acid is 2,2-dimethyl-1,3-dioxane-4,6-dione (Meldrum's acid), uric acid, cyanuric acid, or barbituric acid.

In still another embodiment, the formulation comprises an organic acid that is an imide. In one embodiment, and by way of example, the imide is phthalimide or a substituted phthalimide. In another embodiment, the substituted phthalimide has at least one electron-withdrawing substituent.

In yet another embodiment, the formulation comprises an organic acid that is a hydroxamic acid. In one embodiment, and by way of example, the hydroxamic acid is an aromatic hydroxamic acid containing one hydroxamic functional group bonded directly to an aromatic ring. In one embodiment, the aromatic ring is selected from the group consisting of a benzene ring, a pyridine ring, a naphthalene ring, a quinoline ring, and a biphenyl ring. In still another embodiment, the hydroxamic acid is benzhydroxamic acid. In yet another embodiment, the hydroxamic acid is one containing a hydroxamic functional group separated from an aromatic ring by a chain of 1-4 sp^{a}-hybridized carbon atoms.

In still another embodiment, formulation comprises a hydroxamic acid that is a dihydroxamic acid containing two or more hydroxamic acid functional groups bonded directly to a benzene ring, a pyridine ring, a naphthalene ring, a quinoline ring, a coumarin ring, or a biphenyl ring system.

In other embodiments, the hydroxamic acid contains an aromatic ring that bears an electron donating substituent selected from hydroxy, methoxy, amino, alkylamino, dialkylamino, and alkyl groups.

In other embodiments, the hydroxamic acid is an aliphatic dihydroxamic acid containing 6-10 carbon atoms.

The hydroxamic acid is, in one embodiment, suberohydroxamic acid.

The hydroxamic acid is, in other embodiments, an unsaturated dihydroxamic acid containing 6-10 carbon atoms.

In another embodiment, the formulation comprises an aromatic carboxylic acid that is selected from the group consisting of 3-phenylpropionic acid, cinnamic acid, a hydroxy-derivative of cinnamic acid, a methoxy derivative of cinnamic acid, nicotinic acid, benzoic acid, an amino-derivative of benzoic acid, a methoxy derivative of benzoic acid, and phthalic acid.

In yet another embodiment, the hydroxy-derivative of cinnamic acid is m-coumaric acid or p-coumaric acid.

In yet other embodiments, the p-coumaric acid is trans-p-coumaric acid.

In other embodiments, the methoxy derivative of cinnamic acid is p-methoxycinnamic acid or m-methoxycinnamic acid.

In still other embodiments, the amino-derivative of benzoic acid is o-amino-benzoic acid (anthranilic acid) or 4-aminobenzoic acid (para-aminobenzoic acid; PABA).

In another embodiment, the methoxy derivative of benzoic acid is 4-methoxybenzoic acid (p-anisic acid), o-anisic acid or m-anisic acid.

In one embodiment, the composition is in a dry form. In another embodiment, the composition is in dry form and hydrates in situ when in its environment of use.

In another aspect, a device comprising a composition as described herein is provided. The device is configured for subcutaneous implantation into a mammal.

In another aspect, an implantable device is provided. The device comprises a reservoir comprising a formulation of a small molecule therapeutic agent, the formulation comprising (i) an amount of the small molecule therapeutic agent to provide substantially zero-order release of the small molecule therapeutic agent for a delivery period of at least about 30 days and at a rate that provides a therapeutic effect and (ii) an organic polyprotic acid that (a) maintains a pH of the formulation when hydrated in its environment of use of between 3.0-6.5 for the delivery period; (b) is present at less than or equal to one stoichiometric equivalent, relative to the therapeutic agent, and/or (c) contains a stoichiometric excess of acidic functional groups relative to the therapeutic agent.

In another aspect, an implantable device is provided. The device comprises a reservoir comprising a formulation of a small molecule therapeutic agent, the formulation comprising (i) an amount of the small molecule therapeutic agent to provide substantially zero-order release of the small molecule therapeutic agent for a delivery period of at least about 30 days and at a rate that provides a therapeutic effect and (ii) a mixture of organic acids containing at least one organic polyprotic acid that (a) maintains a pH of the formulation when hydrated in its environment of use of between 3.0-6.5 for the delivery period; (b) contains a total number of acid molecules less than or equal to one stoichiometric equivalent, relative to the therapeutic agent, and/or (c) has a stoichiometric excess of acidic functional groups relative to the therapeutic agent.

In another aspect, an implantable device is provided. The device comprises a reservoir comprising a formulation of a small molecule therapeutic agent, the formulation comprising (i) an amount of the small molecule therapeutic agent to provide substantially zero-order release of the small molecule therapeutic agent for a delivery period of at least about 30 days and at a rate that provides a therapeutic effect and (ii) an organic polyprotic acid that (a) maintains a pH of the formulation when hydrated in its environment of use less than or equal to the pKa of the protonated therapeutic agent; (b) is present at less than or equal to one stoichiometric equivalent, relative to the therapeutic agent, and/or (c) has a stoichiometric excess of acidic functional groups relative to the therapeutic agent.

In another aspect, an implantable device is provided. The device comprises a reservoir comprising a formulation of a small molecule therapeutic agent, the formulation comprising (i) an amount of the small molecule therapeutic agent to provide substantially zero-order release of the small molecule therapeutic agent for a delivery period of at least about 30 days and at a rate that provides a therapeutic effect and (ii) a mixture of organic acids containing at least one organic polyprotic acid that (a) maintains a pH of the formulation when hydrated in its environment of use less than or equal to the pKa of the protonated therapeutic agent; (b) contains a total number of acid molecules less than or equal to one stoichiometric equivalent, relative to the therapeutic agent, and/or (c) has a stoichiometric excess of acidic functional groups relative to the therapeutic agent.

In one embodiment, the formulation is in dry form. In various embodiments, and by way of example, the formulation is a powder, a tablet or a film; or a mixture of two or more powders, tablets, or films.

In another embodiment, the formulation hydrates in the presence of an aqueous solution to form an aqueous suspension. In one embodiment, the aqueous solution is in vivo fluid.

In another embodiment, the small molecule therapeutic agent is released from the device at a rate that provides a therapeutic effect for the period.

In still another embodiment, the organic acid has a water solubility at 25° C. of less than about 20 g/L. In still another embodiment, the organic acid has a water solubility at room temperature between 0.1 and 10 g/L.

In another embodiment, the organic acid has a water solubility at 25° C. of less than about 20 g/L and a pKa between 3 and 6. In another embodiment, the organic acid has a water solubility at room temperature between 0.1 and 10 g/L and a pKa between 3 and 6.

In another embodiment, two or more organic acids, one of which is a polyprotic acid, each with a water solubility of 0.1 to 10 g/L, a molar mass less than 500 grams per mole, and a pKa between 3 and 6 are used in combination.

In another embodiment, two or more monoprotic organic acids are present in the formulation, where each monoprotic acid in the formulation is in an amount that is equal to or less than the molar amount of the therapeutic agent and wherein the two or more monoprotic acids provide a total number of acid hydrogens that exceeds the stoichiometric equivalent for the molar amount of therapeutic agent.

In yet another embodiment, a dendrimer or star polymer capped with acidic functional groups, such as a PAMAM dendrimer capped with carboxymethyl, 3-carbomethoxypyrrolidone, succinamic, or glutaramic acid groups is used.

In yet another embodiment, the organic acid has a melting temperature of greater than about 37° C.

In another aspect, a method for sustained, controlled delivery of a small molecule therapeutic is provided. The method comprises providing a composition or a device as described herein. In some embodiments, the method further comprises administering the device, such as by subcutaneous implantation.

In another aspect, a method for sustained, controlled delivery of an antipsychotic drug is provided, where the method comprises providing a composition or a device as described herein. In some embodiments, the method further comprises administering the device, such as by subcutaneous implantation.

In another aspect, a method to provide maintenance therapy to treat schizophrenia or bipolar disorder is provided, where the method comprises providing a composition or a device as described herein. In some embodiments, the method further comprises administering the device, such as by subcutaneous implantation.

In another aspect, a method to provide maintenance therapy to treat drug addiction is provided, where the method comprises providing a composition or a device as described herein. In some embodiments, the method further comprises administering the device, such as by subcutaneous implantation.

In another aspect, a method to provide maintenance therapy to treat Parkinson's disease or Alzheimer's disease is provided, where the method comprises providing a composition or a device as described herein. In some embodiments, the method further comprises administering the device, such as by subcutaneous implantation.

In another aspect, a method to provide maintenance therapy to treat epilepsy, multiple sclerosis, or amyotrophic lateral sclerosis is provided, where the method providing a composition or a device as described herein. In some embodiments, the method further comprises administering the device, such as by subcutaneous implantation.

In another aspect, a method to provide prophylaxis against malaria is provided, where the method providing a composition or a device as described herein. In some embodiments, the method further comprises administering the device, such as by subcutaneous implantation.

In yet another aspect, a method to treat osteoporosis, breast cancer, or infertility is provided, where the method providing a composition or a device as described herein. In some embodiments, the method further comprises administering the device, such as by subcutaneous implantation.

In addition to the exemplary aspects and embodiments described above, further aspects and embodiments will become apparent by reference to the drawings and by study of the following descriptions.

Additional embodiments of the present methods, devices and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIGS. 1A-1B are illustrations of a drug delivery device, in assembled form (FIG. 1A) and in unassembled form (FIG. 1B).

FIGS. 1C-1F illustrate a portion of a first exemplary drug delivery device, showing the end cap subassembly in cross sectional in assembled form (FIG. 1C) and in an exploded view (FIG. 1D), and in isometric view when assembled (FIG. 1E). FIG. 1F shows an exploded view of the cap subassembly alone. The numbered elements of the subassembly are 1=cap, 2=porous membrane, 3=seal, 4=retention ring, and 5=drug device reservoir.

FIGS. 1G-1K illustrate a portion of a second exemplary drug delivery device, showing the end cap subassembly in cross sectional in assembled form (FIG. 1G) and in an exploded view (FIG. 1H), and in isometric view when assembled (FIG. 1I). FIGS. 1J-1K show an assembled and exploded view of the cap subassembly alone. The numbered elements of the subassembly are 1=cap, 2=porous membrane, 3=seal, 4=drug delivery device reservoir, and 5=retention ring.

FIG. 2 shows the cumulative release of naltrexone (in mg) as a function of time (in days) from drug delivery devices comprising a composition of naltrexone base and a less than equimolar amount of a polyprotic acid suberic acid (x symbols) or a composition of naltrexone base with no polyprotic acid (diamonds).

FIG. 3 shows the weight-normalized plasma levels of naltrexone (in ng/mL) in rats as a function of time, in days, following implantation with a drug delivery device that comprised naltrexone and sebacic acid (a diprotic acid) in a 1:0.6 mole ratio, where one group of devices (“Group 1”, diamonds) had approximately half of the diffusive surface area of a second group of devices (“Group 2”, x symbols). Data are plotted +/−one standard deviation as an indicator of variance.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 mg to 8 mg is stated, it is intended that 2 mg, 3 mg, 4 mg, 5 mg, 6 mg, and 7 mg are also explicitly disclosed, as well as the range of values greater than or equal to 1 mg and the range of values less than or equal to 8 mg.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The compositions of the present disclosure can comprise, consist essentially of, or consist of, the components disclosed.

All percentages, parts and ratios are based upon the total weight of the compositions and all measurements made are at about 25° C., unless otherwise specified.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

The term “treating” is used herein in reference to methods of administration of a small molecule which reduces the frequency of, or delays the onset of, symptoms of a medical condition (e.g., schizophrenia, bi-polar disorder) in a subject relative to a subject not receiving the compound or composition. This can include reversing, reducing, or arresting the symptoms, clinical signs, and underlying pathology of a condition in a manner to improve or stabilize a subject's condition (e.g., controlling schizophrenia symptoms).

The term “polyprotic,” when used to describe an acid, is used herein in reference to molecules that contain either a plurality of acidic functional groups or to molecules that have a single functional group that can react with one or more stoichiometric equivalents of a strong base (for instance, sodium hydroxide) to produce a plurality of salt forms. For instance, the term “polyprotic” may refer to a diprotic acid such as succinic acid, glutaric acid, adipic acid, and the like, or to a triprotic acid such as citric acid, aconitic acid, phosphoric acid, and the like.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Formulations to Enhance the Solubility of a Small Molecule Therapeutic Agent

In one aspect, a composition or formulation in which a small molecule therapeutic agent is solubilized through the use of partially soluble acids to improve delivery of the therapeutic agent from a device or drug delivery platform for a sustained period of time. In one embodiment, the composition is an aqueous suspension or slurry. In another embodiment, the composition is a heterogeneous or nonuniform mixture or solution. The solution or mixture can be, in some embodiments, an aqueous mixture or an aqueous heterogeneous mixture. In another embodiment, the composition is in dry form (e.g., lyophilized, spray dried, desiccated, etc.).

In these various embodiments, the composition comprises a small molecule therapeutic agent that can function as a Bronsted or Lewis base combined with less than or equal to one equivalent of an organic polyprotic acid compound that (i) provides a stoichometric excess of acidic functional groups relative to the therapeutic agent, (ii) has a water solubility between 0.1 and 10 g/L; (iii) has a molecular weight less than 500 grams per mole; and/or (iv) maintains a pH of the suspension in its environment of use that is equal to or below the pKa of the protonated drug for a period of at least about 30 days. In another embodiment, the composition comprises a small molecule therapeutic agent that can function as a Bronsted or Lewis base and more than one monoprotic acid compound, where the more than one monoprotic acid compounds collectively provide a stoichometric excess of acidic functional groups relative to the therapeutic agent. In one embodiment, each monoprotic acid in the more than one monoprotic acid compounds is present in the formulation at equal to or less than a molar amount of the therapeutic agent. In another embodiment, each monoprotic acid in the mixture is in an amount that is equal to or less than the molar amount of the therapeutic agent and wherein the two or more monoprotic acids provide a total number of acid hydrogens that exceeds the stoichiometric equivalent for the molar amount of therapeutic agent.

The composition may additionally comprise an aqueous fluid, for example water, buffer or a water-solvent mixture. In embodiments where the composition is in dry form, the aqueous fluid hydrates the composition in situ in its environment of use.

In other compositions and formulations, the composition comprises a small molecule therapeutic agent that can function as a Bronsted or Lewis base combined with a mixture of organic acids containing at least one organic polyprotic acid compound, such that (i) the sum total of all acid molecules combined with the therapeutic agent is less than or equal to the stoichiometric quantity of therapeutic agent, (ii) the total number of acidic functional groups exceeds the stoichiometric quantity of therapeutic agent present, (iii) of the acidic compounds added to the formulation, greater or equal to 50% of the acids by mass have a water solubility at room temperature of less than about 20 g/L, (iv) of the acidic compounds added to the formulation, greater or equal to 50% of the acids by mass have a molecular weight less than or equal to 500 grams per mole, and/or (v) the pH of the suspension in its environment of use is maintained between 3-6.5 for a period of at least about 30 days. In other compositions and formulations, the composition comprises a small molecule therapeutic agent that can function as a Bronsted or Lewis base combined with less than or equal to one equivalent of an inorganic polyprotic acid that (i) provides a stoichometric excess of acidic hydrogens (i.e., those with a pKa less than or equal to 12) relative to the therapeutic agent, (ii) has a water solubility between 0.1 and 10 g/L; (iii) has a molecular weight less than 500 grams per mole; and/or (iv) maintains a pH of the suspension in its environment of use that is equal to or below the pKa of the protonated drug for a period of at least about 30 days. The composition may additionally comprise an aqueous fluid, for example water, buffer or a water-solvent mixture. In embodiments where the composition is in dry form, the aqueous fluid hydrates the composition in situ in its environment of use.

In other compositions and formulations, the composition comprises a small molecule therapeutic agent that can function as a Bronsted or Lewis base combined with a mixture of organic and inorganic acid compounds, such that (i) the mixture contains at least one polyprotic acid, (ii) the sum total of all acid molecules combined with the therapeutic agent is less than or equal to the stoichiometric quantity of therapeutic agent, (iii) the total number of acidic hydrogens exceeds the stoichiometric quantity of therapeutic agent present, (iv) of the acidic compounds added to the formulation, greater or equal to 50% of the acids by mass have a water solubility at room temperature of less than about 20 g/L, (v) of the acidic compounds added to the formulation, greater or equal to 50% of the acids by mass have a molecular weight less than or equal to 500 grams per mole, and/or (v) the pH of the suspension in its environment of use is maintained between about 3.0-6.5 for a period of at least about 30 days.

In other compositions and formulations, the composition comprises a small molecule therapeutic agent that can function as a Bronsted or Lewis base combined with a dendrimer capped with acidic functional groups. The dendrimer is present in the composition at stoichiometric ratio less than or equal to 0.25 relative to the therapeutic agent. Additionally, the dendrimer (i) provides a stoichometric excess of acidic hydrogens (i.e., those with a pKa less than or equal to 12) relative to the therapeutic agent, and (ii) maintains a pH of the suspension in its environment of use that is equal to or below the pKa of the protonated drug for a period of at least about 30 days. The composition may additionally comprise an aqueous fluid, for example water, buffer or a water-solvent mixture. In embodiments where the composition is in dry form, the aqueous fluid hydrates the composition in situ in its environment of use.

As noted above, the formulations described herein provide solubility of the small molecule therapeutic agent in order to permit delivery for a sustained period. In one embodiment, a sustained period of time intends a period of at least about two weeks to about six months. In another embodiment, a sustained period of time intends a period of at least about two weeks, or at least about three weeks, or at least about four weeks to about six months, or to about four months, or to about three months. In another embodiment, a sustained period of time intends a period of at least about 15 days, or at least about 21 days, or at least about 30 days, or at least about 45 days, or at least about 60 days. In another embodiment, the sustained period of time intends a period of at least about six months, or nine months, or twelve months.

Also as noted above, the formulations described herein enhance the solubility of the small molecule therapeutic in part by maintaining a particular pH range of the formulation in its environment of use for the stated period of time. In one embodiment, the environment of use is in vivo. For example, the formulation may be part of a drug delivery device that is implanted in vivo and several examples of such devices are provided below. In another embodiment, the environment of use is in vitro in a release medium maintained at about 37° C.

The components of the composition, namely the small molecule therapeutic agent and the organic acid compound (also referred to herein as an ‘organic acid’), are now described.

A. Small Molecule Therapeutic Agents

In one embodiment, the compositions comprise a small molecule therapeutic agent that (i) has a water solubility at room temperature of less than 1.0 g/L and (ii) is an organic base. Reference to “small molecule”, in one embodiment, is to a biologically active molecule that has a molecular weight of less than or equal to 2,000 Daltons, and is generally used in the context of a small molecule drug (therapeutic agent) as distinguished from a protein, polypeptide or peptide therapeutic agent. In another embodiment, the small molecule has a molecular weight of less than or equal 1,000 Daltons or less than or equal to 500 Daltons. In other embodiments, the molecular weight of the small molecule is between 10-2000 Daltons, 10-1000 Daltons, 10-500 Daltons, 50-2000 Daltons, 50-1000 Daltons, 50-500 Daltons, 100-2000 Daltons, 100-1000 Daltons, or 100-500 Daltons.

Small molecule therapeutic agents contemplated include, but are not limited to, agents that are weak organic bases (i.e., possessing conjugate acids with pKas between 6 and 9 or between 5 and 9) and a potency such that a 30-60 day dose can be contained in a delivery device implanted into a human.

By way of example, therapeutic agents that include a primary, secondary, or tertiary amine; aniline, amidine, or guanidine; or a nitrogen-bearing heterocyclic ring such as pyridine, quinoline, imidazole, thiazole, triazole, or tetrazole functional group are contemplated as small molecule therapeutic agents that are organic bases. It will be appreciated that therapeutic agents having a structure containing more than one of these functional groups are contemplated. Examples of aniline derivatives include analogues of aniline where the phenyl group is substituted with, for example, a methyl group (toluidine), a halogen such as chlorine (2-chloroaniline, 3-chloroaniline, 4-chloroaniline), an amino group (4-aminobenzoic acid, or 2-aminobenzoic acid, or 3-aminobenzoic acid), a nitro group (e.g., 2-, 3-, or 4-nitroaniline), and many others.

In one embodiment, the small molecule therapeutic agent is an antipsychotic agent. In various embodiments, the antipsychotic agent is selected from risperidone, olanzapine, paliperidone, aripiprazole, brexpiprazole, asenapine, cariprazine, lurasidone, or haloperidol.

In one embodiment, the small molecule therapeutic agent is an opioid agonist or antagonist. In an embodiment, the opioid agonist or antagonist is selected from buprenorphine, naloxone, naltrexone, fentanyl, and meperidine.

In another embodiment, the small molecule therapeutic is an antimigraine drug. In an embodiment, the antimigraine drug is selected from rizatriptan and naratriptan.

In another embodiment, the small molecule therapeutic is an antiemetic drug. In an embodiment, the antiemetic drug is selected from ondansetron and granisetron.

In another embodiment, the small molecule therapeutic is an anticonvulsant. In an embodiment, the anticonvulsant drug is peramanel.

In another embodiment, the small molecule therapeutic is an anti-Parkinsonian agent. In an embodiment, the anti-Parkinsonian agent is selected from pramipexole, ropinirole, cabergoline, and bromocriptine.

In one embodiment, the small molecule therapeutic is a cholinesterase inhibitor. In an embodiment, the cholinesterase inhibitor is selected from such as rivastigmine and donepezil.

In one embodiment, the small molecule therapeutic is a skeletal muscle relaxant. In an embodiment, the skeletal muscle relaxant is tizanidine.

In one embodiment, the small molecule therapeutic is a nicotine agonist or partial agonist. In an embodiment, the nicotine agonist or partial agonist is varenicline.

In one embodiment, the small molecule is an alpha-blocker. In an embodiment, the alpha-blocker is prazosin or terazosin. In one embodiment, the small molecule is a beta blocker, such as metoprolol or propranolol.

In one embodiment, the small molecule is a benzothiazole, such as riluzole.

In one embodiment, the small molecule is a cardiac inotropic agent. In an embodiment, the cardiac inotropic agent is dobutamine.

In one embodiment, the small molecule is an antimalarial agent. In an embodiment, the antimalarial agent is primaquine.

In one embodiment, the small molecule is an immunomodulatory agent. In an embodiment, the immunomodulatory agent is fingolimod.

In one embodiment, the small molecule is an aromatase inhibitor. In an embodiment, the aromatase inhibitor is selected from anastrazole and letrozole.

In one embodiment, the small molecule is an antiestrogen compound. In an embodiment, the antiestrogen compound is selected from tamoxifen and raloxifene.

In one embodiment, the small molecule drug is i) poorly water soluble at physiological pH (˜7.4) and/or ii) functions as a Bronsted or Lewis base. As will be described below, in the presence of an aqueous fluid and less than one stoichiometric equivalent of a polyprotic acid or of two or more monoprotic acids that i) has a solubility in water between 0.1 and 10 g/L or less than or equal to 20 g/L at 25° C., and/or ii) dissolves at least partially in the presence of the therapeutic agent and a physiological buffer, a suspension or slurry is produced with a pH (within the aqueous fraction) approximately equal to or less than the pKa of the protonated therapeutic agent. In other embodiments, the minor stoichiometric fraction of a single polyprotic acid may be substituted with a mixture of acids, including at least one polyprotic acid, in a combined stoichiometric amount less than or equal to that of the therapeutic agent.

In one embodiment, the drug is selected from the groups consisting of buprenorphine, naloxone, naltrexone, fentanyl, and meperidine; rizatriptan and naratriptan; ondansetron and granisetron; peramanel; pramipexole, ropinirole, cabergoline, andbromocriptine; rivastigmine and donepezil; tizanidine; varenicline; prazosin; dobutamine; primaquine; fingolimod; anastrazole and letrozole; tamoxifen and raloxifene. In another embodiment, the drug is selected from the group consisting of buprenorphine, naloxone, naltrexone, fentanyl, meperidine, rizatriptan, naratriptan, ondansetron, granisetron, peramanel, pramipexole, ropinirole, cabergoline, bromocriptine, rivastigmine, donepezil, tizanidine, varenicline, prazosin, dobutamine, primaquine, fingolimod, anastrazole, letrozole, tamoxifen, raloxifene.

B. Organic Acids (Organic Acid Compounds)

The composition, in addition to a small molecule therapeutic agent, comprises a polyprotic acid compound or combination of organic acid compounds, for example a combination that includes at least one polyprotic acid or a combination of two or more monoprotic acid compounds. The polyprotic acid or combination of polyprotic acids and/or monoprotic acids has one or more of the following features: (i) a water solubility at room temperature of between 0.1 and 10 g/L or of less than about 20 g/L; (ii) is present at less than or equal to one stoichiometric equivalent, relative to the therapeutic agent; (iii) provides a stoichiometric excess of acidic hydrogen atoms (with pKa values less than or equal to 12) relative to the therapeutic agent; and/or (iv) maintains a pH of the suspension or solution in its environment of use approximately equal to or less than the pKa of the protonated small molecule therapeutic agent for a period of at least about 30 days. Optionally, a polyprotic acid may be present within a mixture of acidic excipients, such that (i) the total stoichiometric quantity of acid excipient molecules is less than or equal to the amount of therapeutic agent; (ii) the totality of acid excipients provides a stoichiometric excess of acidic hydrogen atoms (with pKa values less than or equal to 12) relative to the therapeutic agent; and/or (iii) the composition maintains the pH of the suspension or solution in its environment of use approximately equal to or less than the pKa of the protonated small molecule therapeutic agent for a period of less than about 30 days. Optionally, more than one or two or more monoprotic acid compounds forms a mixture of acidic excipients, such that (i) the total stoichiometric quantity of acid excipient molecules is less than or equal to the amount of therapeutic agent; (ii) the totality of acid excipients provides a stoichiometric excess of acidic hydrogen atoms (with pKa values less than or equal to 12) relative to the therapeutic agent; and/or (iii) the composition maintains the pH of the suspension or solution in its environment of use approximately equal to or less than the pKa of the protonated small molecule therapeutic agent for a period of less than about 30 days. As described above, the compositions enhance the solubility of the small molecule therapeutic agent, permitting use of the composition in a drug delivery platform that provides sustained release for an extended period of time. Excess acidic hydrogens (relative to the therapeutic agent) intercept physiological buffering species that would otherwise drive hydrolysis of the pharmacologically active salt. Examples of organic acids for use in the compositions are now described.

In a first embodiment, the organic acid is a carboxylic acid. Examples include aromatic carboxylic acids where a carboxylic acid group is bonded directly to an aromatic ring. For example, the aromatic carboxylic acid can have one carboxylic acid group bound to an unsubstituted benzene or pyridine ring. Examples include benzoic acid, picolinic acid, nicotinic acid, or isonicotinic acid. In another example, the aromatic carboxylic acid is one having a benzene ring and one electron-donating group with antioxidant properties. Specific examples include o-anisic acid, m-anisic acid, p-anisic acid, p-aminobenzoic acid (PABA), o-aminobenzoic acid (anthranilic acid), o-toluic acid, m-toluic acid, p-toluic acid and salicylic acid.

In yet another example, the aromatic carboxylic acid is one having a single benzene ring and two electron donating groups with antioxidant properties. A specific example is vanillic acid. In still another example, the aromatic carboxylic acid is one having two or more carboxylic acid groups bonded to a benzene ring. A specific example is phthalic acid.

In another example, the aromatic carboxylic acid is one having one carboxylic acid group bonded to a naphthalene, quinoline, or coumarin ring. Examples include 1-naphthoic acid, 2-naphthoic acid, quinaldic acid, 3-quinolinecarboxylic acid, 4-quinolinecarboxylic acid, 5-quinolinecarboxylic acid, 6-quinolinecarboxylic acid, 7-quinolinecarboxylic acid, and 8-quinolinecarboxylic acid. A further grouping of acids of this type, with one carboxylic acid group bonded to a naphthalene or quinoline ring, include those containing an additional electron-donating group, such as a hydroxy, methoxy, amino, alkylamino, dialkylamino, or alkyl group. Examples of acids in this grouping include 6-hydroxy-2-naphthoic acid, 6-hydroxy-3-naphthoic acid, 8-hydroxy-2-quinolinecarboxylic acid, 8-hydroxy-7-quinolinecarboxylic acid, 7-hydroxycoumarin-3-carboxylic acid, and isomers of each.

In another exemplary embodiment, the carboxylic acid is one having one carboxylic acid group bonded to a biphenyl ring with an electron donating substituent such as a hydroxyl group on the carboxylic acid moiety. Examples include 4′-hydroxy-4-biphenylcarboxylic acid, 4′-hydroxy-2-biphenylcarboxylic acid, 4′-methyl-4-biphenylcarboxylic acid, 4′-methyl-2-biphenylcarboxylic acid, 4′-methoxy-4-biphenylcarboxylic acid, and 4′-methoxy-2-biphenylcarboxylic acid.

In another exemplary embodiment, the acid is a di- or tri-carboxylic acid having two or three carboxylic acid groups bonded to a naphthalene or quinoline ring. Examples include 1,4-naphthalenedicarboxylic acid and 2,6-naphthalenedicarboxylic acid.

In another exemplary embodiment, the carboxylic acid is one having one or two carboxylic acid groups directly bonded to a biphenyl ring system. Examples include 2-phenylbenzoic acid, 3-phenylbenzoic acid, 4-phenylbenzoic acid and diphenic acid.

In another exemplary embodiment, the carboxylic acid is one having a carboxylic acid functional group separated from a benzene, pyridine, naphthalene, quinoline, or coumarin ring by a chain of 1-4 saturated carbon atoms. Examples of acids in this embodiment include phenylacetic acid and 3-phenylpropionic acid. Such an acid may also be modified with one or more electron donating groups such as hydroxy or methoxy, such as 7-hydroxycoumarin-4-acetic acid.

In another exemplary embodiment, the carboxylic acid is an aliphatic dicarboxylic acid with 6-10 carbon atoms, such as adipic acid ((CH₂)₄(COOH)₂), pimelic acid (HO₂C(CH₂)₅CO₂H), suberic acid (HO₂C(CH₂)₆CO₂H), azelaic acid (HO₂C(CH₂)₇CO₂H), and sebacic acid (HO₂C(CH₂)₈CO₂H).

In another exemplary embodiment, the carboxylic acid is an unsaturated or polyunsaturated dicarboxylic acid containing 4-10 carbons. Examples of acids in this embodiment includefumaric acid, trans, trans-muconic acid, cis, trans-muconic acid, and cis, cis-muconic acid.

In another exemplary embodiment, the carboxylic acid is a cis- or trans-cinnamic acid. In one embodiment, the trans-cinnamic acid has one or two electron-donating groups selected from hydroxy, methoxy, amino, alkylamino, dialkylamino, or alkyl groups. Examples include o-coumaric acid, m-coumaric acid, p-coumaric acid, o-methylcinnamic acid, m-methylcinnamic acid, p-methylcinnamic acid, o-methoxycinnamic acid, m-methoxycinnamic acid, and p-methoxycinnamic acid, and ferulic acid.

In another embodiment, the organic acid is a phenol or a naphthol substituted with between about 2-5 electron-withdrawing groups selected from —F, —Cl, —Br, —I, —CN, —CHO (aldehyde), —COR (ketone), and NO₂. Examples include 2,4-dinitrophenol.

In another embodiment, the organic acid is a1,3-dicarbonyl compound containing an acidic CH or NH bond (pKa<8). Examples include 2,2-dimethyl-1,3-dioxane-4,6-dione (Meldrum's acid), uric acid, cyanuric acid, or barbituric acid.

In another embodiment, the organic acid is an imide, such as phthalimide. In one embodiment, the phthalimide is substituted with at least one electron-withdrawing substituent.

In another embodiment, the organic acid is a hydroxamic acid. The hydroxamic acid may be, in some embodiments, an aromatic hydroxamic acid containing one hydroxamic functional group bonded directly to an aromatic ring. The aromatic ring is selected from the group consisting of a benzene ring, a pyridine ring, a naphthalene ring, a quinoline ring, and a biphenyl ring. Examples include benzhydroxamic acid. The hydroxamic acid can also be one containing a hydroxamic functional group separated from an aromatic ring by a chain of 1-4 sp^{a}-hybridized carbon atoms. Dihydroxamic acids containing two or more hydroxamic acid functional groups bonded directly to a benzene, pyridine, naphthalene, quinoline, coumarin, or biphenyl ring system are also contemplated. In addition, substituted derivatives of the hydroxamic acids described above that contain electron donating substituents such as hydroxy, methoxy, amino, alkylamino, dialkylamino, or alkyl groups are contemplated. Also contemplated are aliphatic dihydroxamic acids containing 6-10 carbon atoms, such as suberohydroxamic acid, and unsaturated dihydroxamic acids containing 6-10 carbon atoms.

The organic acids for use in the compositions described herein are preferably those with a water solubility at room temperature between 0.1 and 10 g/L or, alternatively, of less than about 20 g/L. In another embodiment, the organic acids for use in the compositions described herein have a molar mass less than 500 grams per mole. In another embodiment, the acid has a water solubility at room temperature of less than about 20 g/L and a pKa value between about 3 and 6, more preferably a pKa value of between about 3-5.5 or between about 3.5-5.5. In other embodiments, the organic acid is crystalline and has a melting temperature of more than about 37° C.

In other embodiments, the acid is an inorganic polyprotic acid derived from phosphoric acid with a molecular weight less than or equal to 500 grams per mole. Examples of such acids include phosphoric acid, pyrophosphoric acid, sodium dihydrogen phosphate, calcium dihydrogen phosphate, magnesium dihydrogen phosphate, disodium pyrophosphate, monocalcium pyrophosphate, and monomagnesium pyrophosphate.

In other embodiments, the acid is an organic polyprotic acid derived from phosphoric acid with a molecular weight less than or equal to 500 grams per mole. Examples of such acids include methylphosphonic acid, ethylphosphonic acid, propylphosphonic acid, butylphosphonic acid, pentylphosphonic acid, and hexylphosphonic acid.

In other embodiments, the acid is a dendrimer or star polymer capped with acidic functional groups. Examples of such capping groups include methylcarboxylic acid, succinamic, and glutaramic groups.

Compositions comprising less than or equal to one equivalent of a polyprotic acid and a small molecule therapeutic agent are prepared by mixing the acid and the therapeutic agent together in a suitable solvent. Compositions comprising less than or equal to one equivalent of one or more monoprotic acids and a small molecule therapeutic agent are prepared by mixing the acids and the therapeutic agent together in a suitable solvent. In some embodiments, the solvent is an aqueous fluid, such as a buffer or a water-organic solvent mixture.

In some embodiments, the molar amount of organic acid in the composition provides a total number of acid hydrogens that exceeds the stoichiometric equivalent for the molar amount of therapeutic agent in the composition, where the organic acid is two or more monoprotic acids or a monoprotic acid and a polyprotic acid. In embodiments, the stoichiometric equivalent is exceeded by 5%, 10%, 15%, 20%, 25%, 30%, 35%, 45%, 50%, 60%, 70%, 75%, 80%, 90% or 100%. In other embodiments, the organic acid in the formulation provides a total number of acid hydrogens that is 25%, 50%, 75%, or 100% more than the stoichiometric equivalent for the molar amount of therapeutic agent in the composition. In other embodiments, the organic acid in the formulation provides a total number of acid hydrogens that is two-fold, three-fold, four-fold, or five-fold more than the stoichiometric equivalent for the molar amount of therapeutic agent in the composition. It will be appreciated that in any of these embodiments, reference to organic acid intends a single polyprotic acid, a combination of polyprotic acids (e.g., two diprotic acids, two triprotic acids, a diprotic acid and a triportic acid), a combination of two monoprotic acids and/or a combination of a monoprotic acid and a polyprotic acid.

In some embodiments, the molar amount of polyprotic acid in the composition provides a total number of acid hydrogens that exceeds the stoichiometric equivalent for the molar amount of therapeutic agent in the composition. In embodiments, the stoichiometric equivalent is exceeded by 5%, 10%, 15%, 20%, 25%, 30%, 35%, 45%, 50%, 60%, 70%, 75%, 80%, 90% or 100%. In other embodiments, the polyprotic acid in the formulation provides a total number of acid hydrogens that is 25%, 50%, 75%, or 100% more than the stoichiometric equivalent for the molar amount of therapeutic agent in the composition. In other embodiments, the polyprotic acid in the formulation provides a total number of acid hydrogens that is two-fold, three-fold, four-fold, or five-fold more than the stoichiometric equivalent for the molar amount of therapeutic agent in the composition. It will be appreciated that in any of these embodiments, reference to polyprotic acid intends a single polyprotic acid, two or more polyprotic acids (e.g., two diprotic acids, two triprotic acids, a diprotic acid and a triportic acid, two diprotic acids and a triprotic acid, etc.). In one embodiment, the composition excludes a monoprotic acid.

In embodiments where the composition is within a reservoir of a drug delivery device, it will be appreciated that the device when placed in its environment of use is open to the environment of use. That is, the environment of use and the composition in the device are in fluid communication via the pore or porous membrane in the drug delivery device. The compositions described herein include suspensions or slurries containing an steady-state or equilibrium mixture of therapeutic agent salt forms, in addition to a stoichiometric excess of acidic functional groups. In this way, the composition maintains the desired pH of the suspension or heterogeneous solution of between 3.0-6.5, preferably 2.75-5.75, more preferably 2.8-5.6, preferably 2.9-5.6, preferably 3.1-5.5, 3.2-5.5, 3.3-5.5, 3.4-5.5, 3.5-5.5, 3.1-5.4, 3.2-5.4, 3.3-5.4, 3.4-5.4, 3.5-5.4, 3.1-5.3, 3.2-5.3, 3.3-5.3, 3.4-5.3, 3.5-5.3, 3.1-5.2, 3.2-5.2, 3.3-5.2, 3.4-5.2, 3.5-5.2, 3.1-5.1, 3.2-5.1, 3.3-5.1, 3.4-5.1, 3.5-5.1, 3.1-5.0, 3.2-5.0, 3.3-5.0, 3.4-5.0, 3.5-5.0, 3.5-5.5 or 3.5-6.0.

Exemplary Delivery Devices

In another aspect, a drug delivery device for administration of a composition or aqueous suspension as described herein is provided. The drug delivery device can be any implantable device, based on, for example, diffusive, erodible or convective systems, e.g., diffusional systems, osmotic pumps, electro-diffusion systems, electro-osmosis systems, electromechanical systems, and the like. In one embodiment, a controlled drug delivery device is utilized, for controlled, extended delivery of the composition for a period of time. The term “controlled drug delivery device” is meant to encompass any device wherein the release (e.g., rate, timing of release, dosing period) of drug or other desired. substance contained therein is controlled by or determined by the device itself (wholly or in part) and not solely the environment of use. Several non-limiting examples are described.

In one embodiment, the drug delivery device is one having a housing member that defines a reservoir in which the compositions and/or the aqueous suspensions described above are retained. The housing member is of a size and shape that is suitable for implantation into the body. A cylindrical shape is preferable for subcutaneous implantation using a cannula or trocar. The outer diameter of a cylindrically shaped housing member would preferably be in the range of 2 mm to 6 mm and the length in the range of about 10 mm to about 50 mm. The composition or aqueous suspension, in one embodiment, is initially present in a dry form within the reservoir of the device. For example, the aqueous suspension comprising the small molecule therapeutic agent and one or more acidic excipients is prepared and subsequently spray dried, milled or lyophilized to provide a dried form of the aqueous suspension. Alternatively, the individual components in dried form—e.g., the therapeutic agent as a dry solid and an organic acid as a dry solid—are mixed in the correct proportions to provide upon later hydration the desired aqueous suspension. Alternatively, the therapeutic agent and the organic acid may be co-dissolved within a suitable organic solvent such as methanol, ethanol, 1-propanol, 2-propanol, tert-butanol, acetone, 2-butanone, or ethyl acetate, followed by concentration to yield a dried powder suitable for resuspension into an aqueous medium. The dried form of the composition can be tableted or pelleted, loaded in the device and hydrated in situ upon subcutaneous implantation of a device containing the dried composition, or the composition can be hydrated at the time of subcutaneous implantation by a clinician introducing a liquid (e.g. a physiological buffer, isotonic saline, phosphate buffered saline, or aqueous propylene glycol) to a reservoir or matrix containing the composition. The liquid can be provided as part of a kit comprising the drug delivery device and a vial comprising a hydration liquid.

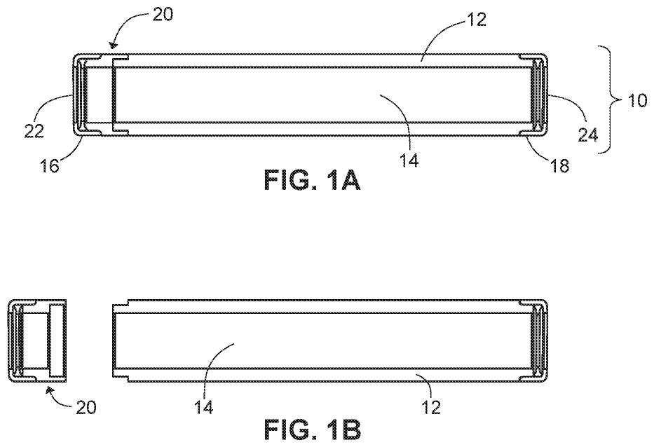

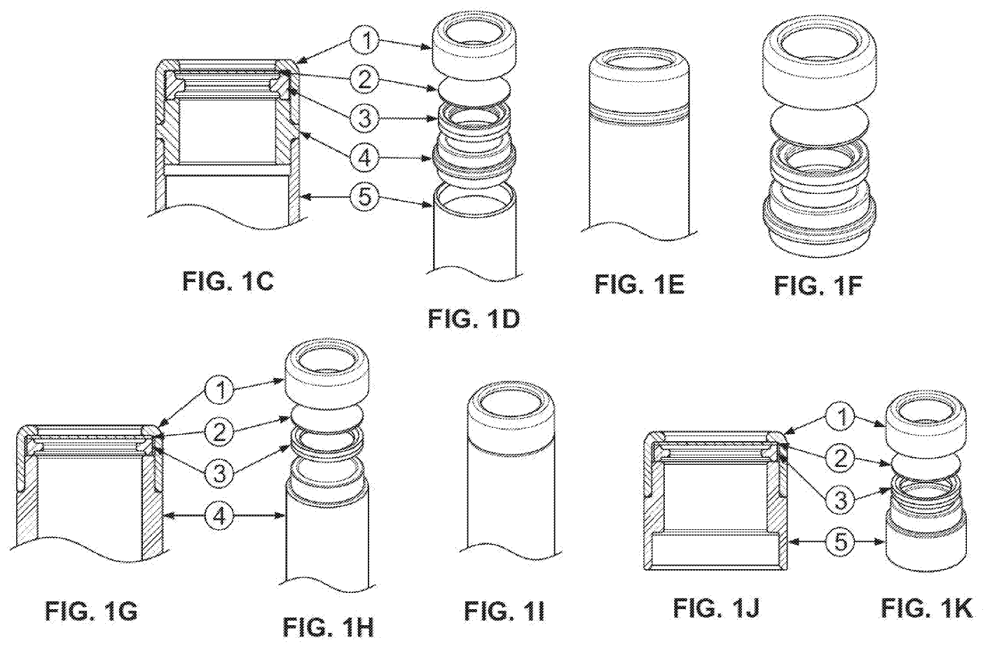

An example of a drug delivery device is provided in FIGS. 1A-1B. FIG. 1A illustrates a device 10, assembled and ready for implantation, in an anatomical compartment of a subject, such as under the skin or in the peritoneal cavity. The device is comprised of a non-erodible housing member 12 that defines an internal compartment or reservoir 14. Contained within the reservoir is a composition or formulation as described herein. Housing member 12 has first and second ends, 16, 18. First end 16 is sealed with a fluid-tight end-cap 20, seen best in FIG. 1B that illustrates device 10 in its unassembled form. End cap 20 may optionally comprise a porous membrane or semi-permeable membrane or porous partition 22. Second end 18 is fitted with a porous membrane, semi-permeable membrane, or porous partition 24.

FIGS. 1C-1K illustrate the end caps and end cap subassembly portions of the exemplary drug delivery devices. The numbered elements of the subassembly illustrated in FIGS. 1C-1F are 1=cap, 2=porous membrane, 3=seal, 4=retention ring, and 5=drug device reservoir. The numbered elements of the subassembly illustrated in FIGS. 1G-1K are 1=cap, 2=porous membrane, 3=seal, 4=drug delivery device reservoir, and 5=retention ring.

The device interior contains a formulation comprising a small molecule drug that is i) poorly water soluble at physiological pH (˜7.4) and/or ii) can function as a Bronsted or Lewis base. The drug, when combined with either i.) a polyprotic acid in an amount that is (a) equal to or less than the molar amount of the therapeutic agent and (b) provides a total number of acid hydrogens that exceeds the stoichiometric equivalent for the molar amount of therapeutic agent, or ii.) a mixture of organic acid excipients that comprises a polyprotic acid or a mixture with two or more monoprotic acid excipients, where the organic acid excipients are present in an that is (a) equal to or less than the molar amount of the therapeutic agent and (b) provides a total number of acid hydrogens that exceeds the stoichiometric equivalent for the molar amount of therapeutic agent. In one embodiment, the therapeutic agent i) has a solubility in water between 0.1 and 10 g/L or of less than or equal to 20 g/L at 25° C., and/or ii) dissolves at least partially in the presence of the drug and a physiological buffer, produces a suspension or slurry with a pH (within the aqueous fraction) approximately equal to or less than the pKa of the protonated drug.

As used herein, the terms “porous membrane” and “porous partition” intend a structural member that has a plurality of pores in the nanometer or micrometer (μm) range, preferably in the 0.1-100 μm or 0.1-200 μm range. The porous partition permits passage of the therapeutic agent in its soluble form from the formulation contained within the reservoir. The porous partition can also permit passage of the organic acid that is part of the formulation in its soluble form. The porous partition in a preferred embodiment retains the therapeutic agent and/or the organic acid in their insoluble forms. That is, the therapeutic agent and/or the organic acid in insoluble form preferably do not pass through the pores of the porous partition. The drug delivery device is described in detail in U.S. 2011/0106006, which is incorporated by reference herein.

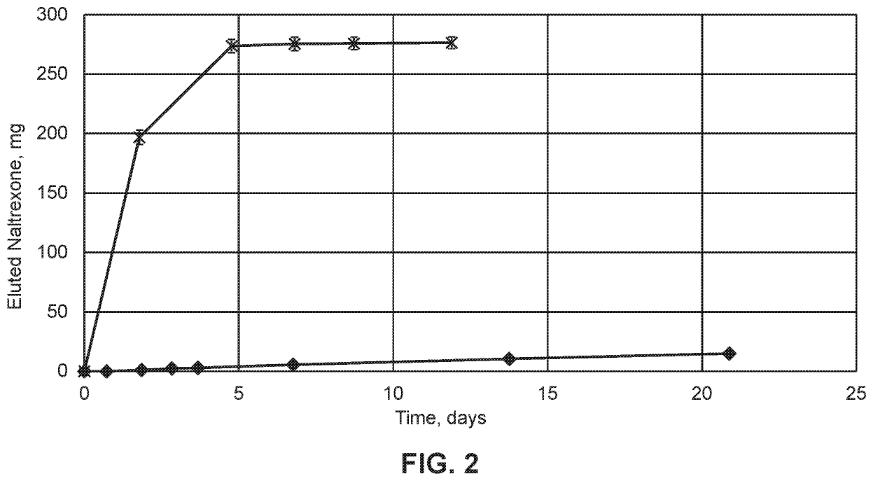

A study was conducted as described in Example 1 where drug delivery devices were prepared. The interior reservoir of the devices were filled with a composition of naltrexone base, a less than molar amount of the diprotic acid suberic acid, polyvinylpyrrolidone and stearic acid. For comparison, devices with a formulation lacking the suberic acid were prepared. In vitro release of naltrexone from the devices was measured, and is shown in FIG. 2. The cumulative release of naltrexone for the devices with the naltrexone: suberic acid formulation (x symbols) released essentially all of the drug in the device over a one week period and at a rate greatly exceeding that of the devices filled with a formulation lacking the polyprotic acid.

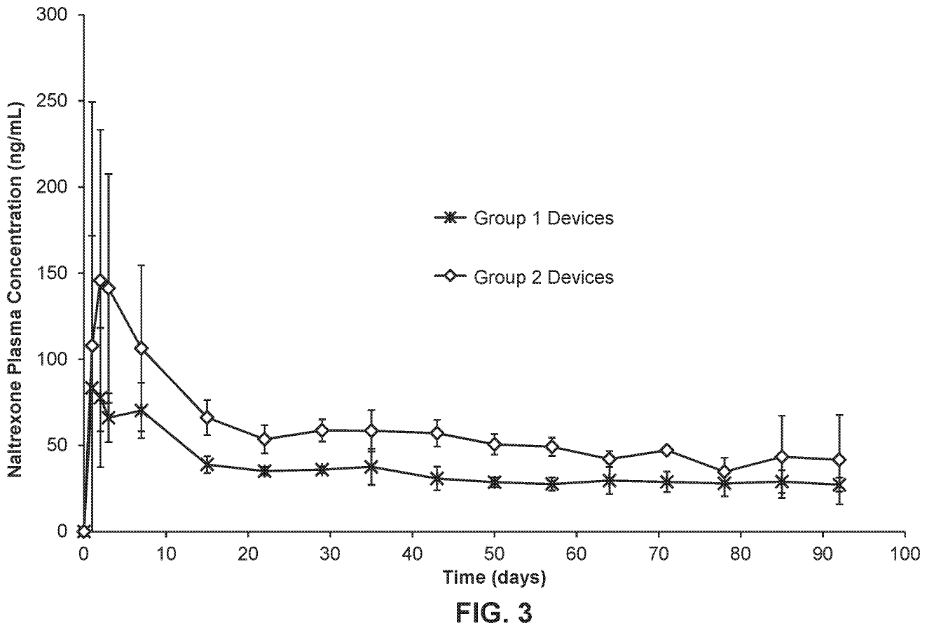

An in vivo study was conducted, as described in Example 2. A solid formulation comprising naltrexone base and sebacic acid (an aliphatic, diprotic acid, also known as decanedioc acid) in a drug:organic acid molar ratio of 1:0.6, along with a binder and a tableting lubricant was prepared. The formulation was compressed into tablets which were inserted into drug delivery devices fitted with a porous membrane. One group of devices (“Group 1”) devices had approximately half of the diffusive surface area of a second group of device (“Group 2”). The devices were implanted into rats and blood samples and animal weights were obtained at time points over a period of approximately 3 months. The concentration of naltrexone in the plasma of each sample was determined and normalized by the individual animal weight. The weight-normalized plasma levels of naltrexone (in ng/mL) are shown in FIG. 3 for the Group 1 (diamonds) and “Group 2” (x symbols) devices. Both Group 1 and Group 2 devices released naltrexone at a relatively constant rate following an initial 1-2 week burst period. The devices were recovered from each animal after the release period, and were subjected to mass balance analysis to calculate an average output rate of drug. Devices from Group 1 (with 25% diffusive surface area) eluted naltrexone at an average rate of 1.8 mg/day. Devices from Group 2 (with a 50% diffusive surface area) eluted naltrexone at an average rate of approximately 3.1 mg/day.

Accordingly, in one embodiment, a formulation and a device for delivery of a therapeutic agent are provided. The therapeutic agent (i) has a water solubility at room temperature of less than 1.0 g/L and (ii) is an organic base. The therapeutic agent is present in the formulation or the device in an amount sufficient to provide a therapeutic effect for a delivery period of at least about 30 days or for at least about 60 days. The formulation also comprises either (i) a polyprotic acid, or (ii) a mixture of acidic excipients including at least one polyprotic acid in an amount that is (a) equal to or less than the molar amount of the therapeutic agent and (b) provides a total number of acid hydrogens that exceeds the stoichiometric equivalent for the molar amount of therapeutic agent. In one embodiment, the organic acid excipient maintains a pH of the formulation when hydrated in its environment of use of between 3.0-11.5 or between about 3.0-6.5 for the delivery period.

In one embodiment, a formulation comprising a small molecule therapeutic agent (also referred to herein as “drug” or “therapeutic agent”) and a polyprotic acid or mixture of acids containing at least one polyprotic acid, provides an increase in the release rate of the small molecule therapeutic agent of at least 10%, 15%, 20%, 25%, 30%, 35%, 40% or 50% compared to a formulation of the small molecule therapeutic agent with no acidic excipients. In one embodiment, the increased rate of release is for a period of at least 14 days, at least 2 weeks, at least 30 days or at least 45 days or at least 60 days or at least 90 days or at least 180 days. In another embodiment, the increased rate of release approaches zero-order kinetic release for the period.

Drug delivery devices other than the one specifically described herein, which is merely exemplary, are known in the art. The compositions described herein are useful for a variety of devices, including those comprise a drug reservoir for retaining the small molecule therapeutic agent and polyprotic acid formulation and those that have a substrate or matrix that can hold or contain the formulation. Controlled drug release devices suitable for use in the present invention generally can provide for delivery of the drug from the device at a selected or otherwise patterned amount and/or rate to a selected site in the subject. The drug delivery device must be capable of containing an amount of the formulation to provide a therapeutically effective amount of the small molecule for the period of therapy. The period of delivery will vary according to the therapeutic agent, the condition being treated, and the individual patient. In one embodiment, the period of delivery, also referred to herein as a sustained period of time, intends a period of at least about two weeks to about six months. In another embodiment, a sustained period of time intends a period of at least about two weeks, or at least about three weeks, or at least about four weeks to about six months, or to about four months, or to about three months. In another embodiment, a sustained period of time intends a period of at least about 15 days, or at least about 21 days, or at least about 30 days, or at least about 45 days, or at least about 60 days. In other embodiments, the period of time is from about 2 hours to about 72 hours, from about 4 hours to about 36 hours, from about 12 hours to about 24 hours, from about 2 days to about 30 days, from about 5 days to about 20 days, from about 7 days or more, from about 10 days or more, from about 100 days or more; from about 1 week to about 4 weeks, from about 1 month to about 24 months, from about 2 months to about 12 months, from about 3 months to about 9 months, from about 1 month or more, from about 2 months or more, or from about 6 months or more.

Accordingly, in another aspect, an implantable device is contemplated. The device comprises a reservoir comprising a formulation of a small molecule therapeutic agent, the formulation comprising (i) an amount of the therapeutic agent to provide substantially zero-order release of the therapeutic agent for a delivery period of at least about 30 days and at a rate that provides a therapeutic effect and (ii) a polyprotic acid, or mixture of acids containing at least one polyprotic acid; and (c) the pH of the formulation when hydrated in its environment of use is maintained at a value of between 3.0-6.0 for the delivery period. In one embodiment, the formulation comprising a small molecule therapeutic agent and a polyprotic acid is in a dry form. For example, the dry formulation may be present in the reservoir of a device as a powder, a tablet or a film. The device when in use, in vitro or in vivo, imbibes fluid from the surrounding environment to hydrate the dry formulation, thus forming in situ an aqueous suspension. The suspension contains an equilibrium or steady-state mixture of the therapeutic agent as a mixture of salt forms. The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are preferred because of convenience in implantation and removal of the drug delivery device. Exemplary subcutaneous delivery sites include under the skin of the arm, shoulder, neck, back, or leg. Sites within a body cavity are also suitable implantation sites. Methods for implanting or otherwise positioning drug delivery devices for subcutaneous delivery of a drug are well known in the art. In general, placement of the drug delivery device will be accomplished using methods and tools that are well known in the art, and performed under aseptic conditions with at least some local or general anesthesia administered to the subject.

Methods of Treatment

In other aspects, methods of treatment using the compositions and devices described herein are contemplated. In one embodiment, a method for sustained, controlled delivery of a therapeutic agent is contemplated, where a composition or a delivery device comprising a formulation of the therapeutic agent and a polyprotic acid (or mixture of acids including at least one polyprotic acid) in less than a stoichiometric amount relative to the therapeutic agent is provided. In one embodiment, the therapeutic agent is an antipsychotic drug used to treat bipolar disorder or schizophrenia. Exemplary agents include risperidone, olanzapine, paliperidone, aripiprazole, brexpiprazole, asenapine, cariprazine, lurasidone, and haloperidol. In one embodiment, the therapeutic agent is an opioid agonist or antagonist, useful for pain relief. Exemplary agents are buprenorphine, naloxone, naltrexone, fentanyl, or meperidine. In another embodiment, the therapeutic agent is an antimigraine drug, such as rizatriptan or naratriptan. In other embodiments, the therapeutic agent is anticonvulsant, such as peramanel, an anti-Parkinsonian agent, such as pramipexole, ropinirole, cabergoline, or bromocriptine, a cholinesterase inhibitor, such as rivastigmine or donepezil, a skeletal muscle relaxant such as tizanidine, a nicotine agonist or partial agonist, such as varenicline, an alpha-blocker such as prazosin, a beta-blocker such as metoprolol, a cardiac inotropic agent such as dobutamine, an antimalarial such as primaquine, an immunomodulator such is fingolimod, an aromatase inhibitor such as anastrazole or letrozole, or an antiestrogen compound such as tamoxifen or raloxifene.

In another embodiment, a method for maintaining therapeutic plasma levels of a therapeutic agent described herein is contemplated, thus delaying relapse for stable, previously medicated patients for at least about 4 weeks is contemplated.

Based on the foregoing, the compositions described herein comprised of a small molecule therapeutic agent and a polyprotic acid provide release of the therapeutic agent for an extended period of time—for example, for at least about 14 days, 21 days, 30 days, 60 days, 90 days, or 120 days—at an essentially constant rate that approaches essentially zero-order release kinetics for the period. The composition comprises the therapeutic agent in an amount sufficient for a therapeutic dose of the agent for the period, and an equimolar or less than equimolar amount of a polyprotic acid (or, alternatively, a mixture of acidic excipients comprising at least one polyprotic acid, where each acidic compound in the mixture of excipients is present at equal to or less than the molar amount of therapeutic agent, yet provides a total number of acid hydrogen that exceeds the stoichiometric equivalent for the molar amount of therapeutic agent) to maintain a concentration of the protonated therapeutic agent at or near its saturation concentration in the hydrated composition for the period. The near-saturated concentration of protonated drug is with respect to the aqueous phase of the composition at, for example, room temperature or at human body temperature. The composition is, in some embodiments, retained in a drug delivery system (or device) and when placed in an environment of use (such as an in vivo environment such as a subcutaneous implantation site, e.g., plasma or interstitial fluid with a constant pH ˜7.4) produces an essentially constant concentration gradient between the device interior and its environment of use that facilitates an essentially constant release rate (substantially zero-order kinetics) of the therapeutic agent over an extended period of time.

### III. Examples

The following examples are illustrative in nature and are in no way intended to be limiting.

### Example 1

### Formulation Comprising Naltrexone as a Small Molecule Therapeutic Agent and a Polyprotic Organic Acid

A composition of naltrexone base (62.2% by mass, approximately 280 mg/device), suberic acid (23.8% by mass; 0.75 molar amount relative to the drug), polyvinylpyrrolidone (12% by mass) and stearic acid (2% by mass) were compounded into compressed pellets. A comparative formulation of naltrexone base (86% by mass, approximately 385 mg/device), polyvinylpyrrolidone (12% by mass) and stearic acid (2% by mass) was also compounded into compressed pellets, to serve as a control. The formulations were loaded into delivery devices equipped with 0.1 micron polyvinylidene fluoride (DURAPORE®) membranes. Devices were vacuum back-filled with phosphate buffer and transferred to jars containing a volume (˜100 mL) of the same buffer. The sealed jars were then incubated at 37° C., and small aliquots (˜500 μL) of receiving buffer were withdrawn at selected time points to quantify the released drug by high pressure liquid chromatography (HPLC). Release of naltrexone is shown in FIG. 2, where the cumulative release of naltrexone (in mg) for the devices (n=3) with the comparative control formulation (diamonds) and for the devices (n=2) with the naltrexone:suberic acid formulation (x symbols) is plotted against time (in days). Error bars reflect the standard deviation for the control systems, and minimum/maximum values for the active systems. Relative to the control, devices loaded with the formulation comprised of drug and polyprotic acid released substantially all of the loaded drug over the span of one week at a rate greatly exceeding that of the control systems.

### Example 2

### In Vivo Evaluation of Drug Delivery Devices Comprising Naltrexone as a Small Molecule Therapeutic Agent and Sebacic Acid

A solid formulation comprising naltrexone base and sebacic acid in a drug:organic acid molar ratio of 1:0.6 was prepared by blending the drug and organic acid with polyvinylpyrrolidone (5 wt %) as a binder and stearic acid (2 wt %) as a lubricant. The mixture was compressed into tablets.

Drug delivery devices were assembled by first sealing one end of a cylindrical reservoir with a solid PEEK cap and filling each resulting tube with the aforementioned tablets. Each device received approximately 450 mg of solid formulation before the second open end was sealed with a cap fitted with a 0.1 micron polyvinylidene fluoride (DURAPORE®) membrane. Two separate device versions were constructed by incorporating washers into the cap assembly to limit the diffusive surface area per device, with one version having a diffusive surface area approximately 25% of the total surface area of the membrane, and the second version have a diffusive surface area approximately 50% of the total surface area of the membrane. All devices were terminally sterilized and vacuum back-filled with sterile saline before being implanted into male Sprague-Dawley rats (n=4). Plasma samples and animal weights were obtained at protocol-prescribed time points over a period of approximately 3 months, and plasma concentrations of naltrexone were obtained by liquid chromatography/mass spectroscopy. Results are shown in FIG. 3. The devices were recovered from each animal after the release period, and were subjected to mass balance analysis to calculate an average output rate of drug. Devices from Group 1 (with 25% diffusive surface area) eluted naltrexone at an average rate of 1.8 mg/day. Devices from Group 2 (with a 50% diffusive surface area) eluted naltrexone at an average rate of approximately 3.1 mg/day.

1-78. (canceled)

79. A composition, comprising:
a molar amount of a therapeutic agent that (i) has a water solubility at room temperature of less than 1.0 g/L and (ii) is an organic base, and
a polyprotic acid in an amount that (i) is equal to or less than the molar amount of the therapeutic agent and (ii) provides a total number of acid hydrogens that exceeds the stoichiometric equivalent for the molar amount of therapeutic agent.

80. The composition of claim 79, wherein the polyprotic acid is an organic polyprotic acid or an inorganic polyprotic acid.

81. The composition of claim 79, wherein the polyprotic acid is selected from the group consisting of a diprotic aliphatic acid containing 4-12 carbon atoms, a triprotic aliphatic acid containing 4-12 carbon atoms, an inorganic diprotic acid, an inorganic triprotic acid, and a tetraprotic acid.

82. The composition of claim 81, wherein the polyprotic acid is selected from succinic, glutaric, adipic, pimelic, suberic, azelic, and sebacic acid.

83. The composition of claim 81, wherein the polyprotic acid is selected from phosphoric acid, pyrophosphoric acid and a salt formed by partial neutralization of pyrophosphoric acid.

84. The composition of claim 79, wherein the polyprotic acid is an unsaturated diprotic acid.

85. The composition of claim 84, wherein the polyprotic acid is selected from maleic, fumaric, glutaconic, traumatic, muconic, citraconic, mesaconic, or itaconic acid.

86. The composition of claim 79, wherein the polyprotic acid is a diprotic acid bearing an aromatic ring.

87. The composition of claim 86, where the polyprotic acid is phthalic acid, isophthalic acid, or terephthalic acid.

88. The composition of claim 79, wherein polyprotic acid is selected from a biphenyl 4,4′-dicarboxylic acid, a regioisomer of a biphenyl 4,4′-dicarboxylic acid, a polyprotic acid with two carboxylic acid groups bound to a naphthalene or quinoline ring, and an alkyl phosphonic acid.

89. The composition of claim 88, where the polyprotic acid is methylphosphonic acid, ethylphosphonic acid, propylphosphonic acid, butylphosphonic acid, pentylphosphonic acid, hexylphosphonic acid, heptylphosphonic acid, or octylphosphonic acid.

90. The composition of claim 79, wherein the composition comprises a hydroxamic acid.

91. The composition of claim 90, wherein the hydroxamic acid is benzhydroxamic acid, suberohydroxamic acid, an unsaturated polyhydroxamic acid containing 6-10 carbon atoms, or an aliphatic polyhydroxamic acid containing 6-10 carbon atoms

92. The composition of claim 79, wherein the therapeutic agent is selected from the group consisting of risperidone, paliperidone, olanzapine, aripiprazole, brexpiprazole, asenapine, cariprazine, lurasidone, haloperidol, buprenorphine, naloxone, naltrexone, fentanyl, meperidine, rizatriptan, naratriptan, ondansetron, granisetron, rivastigmine, donepezil, peramanel, tizanidine, varenicline, prazosin, dobutamine, primaquine, fingolimod, anastrazole, letrozole, tamoxifen, raloxifene, pramipexole, ropinirole, cabergoline, and bromocriptine.

93. A composition, comprising:
a molar amount of a therapeutic agent that (i) has a water solubility at room temperature of less than 1.0 g/L and (ii) is an organic base, and
a mixture of two or more acids, wherein the mixture comprises at least one polyprotic acid in an amount that (i) is equal to or less than the molar amount of the therapeutic agent and (ii) provides a total number of acid hydrogens that exceeds the stoichiometric equivalent for the molar amount of therapeutic agent.

94. The composition of claim 93, wherein the polyprotic acid is an organic polyprotic acid or an inorganic polyprotic acid.

95. A composition, comprising:
a molar amount of a therapeutic agent that (i) has a water solubility at room temperature of less than 1.0 g/L and (ii) is an organic base, and
a mixture of two or more monoprotic acids, wherein each monoprotic acid in the mixture is in an amount that is equal to or less than the molar amount of the therapeutic agent and wherein the two or more monoprotic acids provide a total number of acid hydrogens that exceeds the stoichiometric equivalent for the molar amount of therapeutic agent.

96. The composition of claim 95, wherein the mixture contains a carboxylic acid having a carboxylic acid group bound to an unsubstituted benzene or pyridine ring, a carboxylic acid having a benzene ring and one electron-donating group with antioxidant properties, or a carboxylic acid having one benzene ring and two electron donating groups with antioxidant properties.

97. The composition of claim 96, wherein the carboxylic acid is selected from the group consisting of benzoic acid, picolinic acid, nicotinic acid, isonicotinic acid, o-anisic acid, m-anisic acid, p-anisic acid, p-aminobenzoic acid (PABA), o-aminobenzoic acid (anthranilic acid), o-toluic acid, m-toluic acid, p-toluic acid, salicylic acid, vanillic acid, 1-naphthoic acid, 2-naphthoic acid, quinaldic acid, 3-quinolinecarboxylic acid, 4-quinolinecarboxylic acid, 5-quinolinecarboxylic acid, 6-quinolinecarboxylic acid, 7-quinolinecarboxylic acid, 8-quinolinecarboxylic acid, 6-hydroxy-2-naphthoic acid, 6-hydroxy-3-naphthoic acid, 8-hydroxy-2-quinolinecarboxylic acid, 8-hydroxy-7-quinolinecarboxylic acid, 2-phenylbenzoic acid, 3-phenylbenzoic acid, 4-phenylbenzoic acid, diphenic acid, 4′-hydroxy-4-biphenylcarboxylic acid, 4′-hydroxy-2-biphenylcarboxylic acid, 4′-methyl-4-biphenylcarboxylic acid, 4′-methyl-2-biphenylcarboxylic acid, 4′-methoxy-4-biphenylcarboxylic acid, and 4′-methoxy-2-biphenylcarboxylic acid, cis-cinnamic acid, and a trans-cinnamic acid.

98. The composition of claim 97, wherein the mixture comprises a substituted trans-cinnamic acid selected from the group consisting o-coumaric acid, m-coumaric acid, p-coumaric acid, o-methylcinnamic acid, m-methylcinnamic acid, p-methylcinnamic acid; o-methoxycinnamic acid, m-methoxycinnamic acid, and p-methoxycinnamic acid, and ferulic acid.

99. A composition, comprising:
a molar amount of a therapeutic agent that (i) has a water solubility at room temperature of less than 1.0 g/L and (ii) is an organic base, and
a dendrimer capped with an acidic functional group, the dendrimer in an amount that (i) is between about 0.25-1.0 of the molar amount of therapeutic agent and (ii) provides a total number of acid hydrogens that exceeds the stoichiometric equivalent for the molar amount of therapeutic agent.

100. The composition of claim 99, where the dendrimer is capped with carboxymethyl, succinyl, glutaryl, adipyl, pimelyl, suberyl, azelyl, sebacyl, or phthalyl groups, or wherein the dendrimeric is capped with a plurality of hydroxamic acid functional groups.

101. A device, comprising: a composition according claim 1, wherein the device is configured for subcutaneous implantation into a mammal.

102. A method for sustained, controlled delivery of a therapeutic agent, comprising: providing a device according to claim 101.
